(19) United States
(12) Patent Application Publication
(10) Pub. No.: US 2009/0220934 A1
(43) Pub. Date: Sep. 3, 2009

(54) METHODS AND PROCESSES OF CONTROLLING FERMENTATION

(76) Inventors: Darrel Manoosingh, Winnipeg (CA); Denis Bosc, Winnipeg (CA)

Correspondence Address: BERESKIN AND PARR LLP/S.E.N.C.R.L., s.r.l., 40 KING STREET WEST, BOX 401, TORONTO, ON, M5H 3Y2, CA

(21) Appl. No.: 12/092,774

(22) PCT Filed: Nov. 7, 2006
(86) PCT No.: PCT/CA2006/001819

§ 371 Date: Oct. 7, 2008

Related U.S. Application Data
(60) Provisional application No. 60/733,795, filed on Nov. 7, 2005.

(51) Int. Cl. C12Q 3/00; C12M 1/36

(52) U.S. Cl. 435/3; 435/286.1

(57) ABSTRACT

The present invention relates to methods and processes for controlling fermentation. Two proportional feedback control loops are implemented in a cascade. One control loop is used to maintain the pH by adjusting the dissolved oxygen-stat setpoint. The other control loop is used to maintain the dissolved oxygen by regulating the nutrient feed rate using the newly adjusted dissolved oxygen-set point.

### FIELD OF THE INVENTION

The invention relates to novel methods and processes to control the feeding of cells during fermentation.

### BACKGROUND OF THE INVENTION

Fermentation is an important means of producing products in the biotechnology industry. For example, a number of enzymes, antibiotics, biochemicals, diagnostics and therapeutics are mass-produced in a fermentation production facility. Commonly, the fermentation is done in an aqueous state by fed-batch, continuous or cell-recycling continuous cultures.

A major objective of fermentation is to maximize productivity of the cells in culture. Productivity usually increases as the density of the cells increase. However, high density cultures have a variety of problems including solubility of solid and gaseous substrates in watery media, limitation and/or inhibition of substrates with respect to growth, instability and volatility of substrates and products, accumulation of products or metabolic by-products to a growth-inhibitory level, degradation of products, high evolution rates of CO₂ and heat, high oxygen demand as well as the increasing viscosity of the medium in very dense cultures (Risenberg and Guthke, 1999). Two of the most commonly measured variables in a fermentation system are the dissolved oxygen (DO) concentration and the pH of the fermentation medium. These are key indicators of cellular physiology.

There have been strategies developed to tackle some of these problems. For example Korz et al 1994 describe a fed-batch technique using a pre-determined constant feeding rate that prevents the accumulation of acetic acid. (Korz et al 1995), Chen et al 1997 describe the control of fermentation through alterations in both the nutrient feed rate and the agitation speed (Chen et al 1997). Other approaches include increasing the oxygen input over time or by direct control of the pH. For review see Reisenberg and Guthke 1999. However, fed-batch fermentations that require complex or custom algorithms are often difficult to implement, validate and transfer into manufacturing.

### SUMMARY OF THE INVENTION

The present inventors have developed novel methods and processes of controlling the feeding of cells in a fermentation system through dependence on dissolved oxygen levels.

Accordingly, the present invention provides a method for controlling the feeding of cells in a fermentation system during an induction phase, said method comprising:

(a) measuring dissolved oxygen (DO) and pH levels in the fermentation system;

(b) (i) increasing a DO-stat setpoint when the pH falls below a pH-stat setpoint or (ii) decreasing a DO-stat setpoint when the pH increases above a pH-stat setpoint; and

(c) feeding the cells with nutrient feed when the DO level goes above the DO-stat setpoint.

In a specific embodiment, the present invention provides a method for controlling the feeding of cells in a fermentation system comprising a growth phase and an induction phase, said method comprising:

(1) adding cells and culture media to a fermentation system;

(2) measuring the dissolved oxygen (DO) and pH levels during the growth phase;

(3) increasing the airflow and stirrer rate of the system to a maximum value when the DO reaches a predetermined value;

(4) adding a first nutrient feed to the system when there is an increase in the DO level and/or an increase in the pH level;

(5) ceasing the first nutrient feed when the DO levels are reduced wherein a DO-stat setpoint is determined by adding a preset value to the lowest DO level measured during the growth phase;

(6) measuring dissolved oxygen (DO) and pH levels in the fermentation system during the induction phase;

(7) (i) increasing the DO-stat setpoint when the pH falls below the pH-stat setpoint or (ii) decreasing the DO-stat setpoint when the pH increases above the pH-stat setpoint; and

(8) feeding the cells with a second nutrient feed when the DO level goes above the DO-stat setpoint.

One embodiment of the invention is a method of controlling fermentation, comprising the steps of:
(a) measuring pH of a fermentation medium, wherein when the pH is equal to or less than a pH-stat setpoint, a dissolved oxygen (DO)-stat setpoint is increased; and
(b) measuring DO of the fermentation medium, wherein when the DO is equal to or greater than the DO-stat setpoint, nutrient feed is added to the fermentation medium.
   wherein steps (a) to (b) are repeated until fermentation is complete.

Another embodiment of the invention is a process for conducting fermentation, comprising the steps of:

maintaining a pH of a fermentation medium at about a setpoint by changing the DO-stat setpoint, wherein the change in the DO-stat setpoint is inversely proportional to the difference between the measured pH and the pH-stat setpoint, and

maintaining the DO of the fermentation medium at about a setpoint by changing a nutrient feed rate, and wherein the change in the nutrient feed rate is proportional to the difference between the current DO-stat setpoint and the last DO-stat setpoint.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1a is a graph showing pH and % DO levels over 46 hours fermentation in 1200 L Fed-Batch run.

FIG. 1b is an expanded section of the graph of FIG. 1a showing changes in % DO controlling feed intervals.

FIG. 2 is an algorithm of the automated process.

### DETAILED DESCRIPTION OF THE INVENTION

The invention provides improved methods and processes of controlling fermentation in order to maximize productivity of the cells in culture.

### (A) DEFINITIONS

As used herein, “fermentation” refers to culturing cells. There are typically two types of fermentation: solid/semisolid-state and aqueous fermentation. Thus, the fermentation medium can be a solid or semi-solid, or a liquid. In one embodiment of the invention, aqueous fermentation is used.

As used herein, “fermentation system” includes culturing cells in fed-batch, continuous or cell recycling continuous cultures. In one embodiment of the invention, the fermentation is aerobic. In further embodiment of the invention, the fermentation system is fed-batch.

The term “fed-batch fermentation” as used herein, refers to a fermentation system, wherein substances, such as nutrients, are added to the culture system but nothing is withdrawn except for samples to monitor progress.

As used herein “phases of fermentation” include growth phase and induction phase of the cell culture. Typically, commercial production of products using fermentation includes 2 phases of fermentation: (1) growth and (2) induction. During the growth phase, the cells are cultured in conditions that promote the growth of the cells. While during the induction phase, the cells are cultured in conditions that promote production of the product of interest, which is produced by the cells.

As used herein “cells” include, but are not limited to, bacteria, yeast and eukaryotic cells. The cells can produce the products of interest either naturally or through genetic engineering or recombinant DNA technology. The products of interest include without limitation, proteins, carbohydrates, lipids, and organic compounds. For example, the product of interest can be a soluble protein that is produced by the cell.

The terms “nutrient” or “nutrient feed” as used herein includes without limitation, carbon sources (such as glucose, glycerol, other carbohydrates, and other nutrient solutions containing one or a combination of these sugars), nitrogen sources, precursors, and vitamins and minerals.

The term “pH-stat” as used herein refers to a strategy to control fermentation or cell growth using pH measurements of the fermentation medium. Typically, the pH of the fermentation medium is measured and maintained at a defined setpoint. The term “pH-stat setpoint” as used herein refers to a defined pH value that is the optimal pH for a particular fermentation system.

The term “DO-stat” as used herein refers to a strategy to control fermentation of cell growth by measuring the DO concentration in the fermentation medium. The term “DO-stat setpoint” as used herein refers to a defined DO concentration value. The DO-stat setpoint is generally calculated during the growth phase and is defined as the lowest DO level detected during the growth phase plus a preset value (e.g. 5-20%) while maintaining the pH of the culture at approximately the pH-stat setpoint.

### (B) METHODS

The present invention provides a method for controlling the feeding of cells in a fermentation system during an induction phase, said method comprising:

(a) measuring dissolved oxygen (DO) and pH levels in the fermentation system;

(b) (i) increasing a DO-stat setpoint when the pH falls below a pH-stat setpoint or (ii) decreasing a DO-stat setpoint when the pH increases above a pH-stat setpoint; and

(c) feeding the cells with nutrient feed when the DO level goes above the DO-stat setpoint.

The dissolved oxygen and pH levels can be measured using techniques known in the art, for example, using commercially available probes. These levels are generally taken at regular intervals for example once every hour. Step (b) can be repeated each time there is a change in the pH levels.

The nutrient feed used can be any feed containing a carbon source such as glucose, glycerol etc. and a nitrogen source as previously described. In one embodiment, the nutrient feed comprises glycerol, yeast extract and tryptone. In the induction phase, the nutrient feed may also contain an inducer that induces the expression of the desired protein by the cells. The method of induction depends on the promoter controlling the expression of the protein of interest. For example, there are temperature, pH, and drug inducible promoters such as metallothionine promoters, the heat shock inducible promoters or the tetracycline inducible promoters. In one embodiment, the inducer is L-arabinose.

The pH-stat setpoint is a defined value that is determined for each fermentation system and depends on many factors including, but not limited to, size of reaction vessel, type and concentration of cells being cultured, type of protein being produced, culture medium used, and type of nutrient feed. For culturing bacteria such as E. coli, the pH-stat setpoint is generally 7.0-7.5, preferably 7.1-7.3. Table 2 provides pH-stat setpoint ranges for cells typically used in fermentation.

The pH level can be controlled using a pH-controlling agent, such as a base to increase pH and an acid to decrease pH. In one embodiment of the invention, a pH controlling agent is used to modify the pH of the fermentation medium. In another embodiment of the invention, a pH controlling agent is not used to modify the pH of the fermentation medium. Instead, pH is maintained by adjusting the DO-stat setpoint which controls feeding.

The DO-stat setpoint is a defined value that is determined for each fermentation system. The DO-stat setpoint is generally calculated by adding a preset value to the lowest DO level detected during the growth phase while maintaining the pH of the culture at approximately the pH-stat setpoint. The preset value will be determined for each fermentation system. One of skill in the art can determine the optimum preset value to be added to the lowest % DO value in order to determine the DO-stat setpoint. If the preset value that is added to the lowest % DO is too high, then the addition of feed may not be triggered during the induction phase. If the preset value is too low, then the addition of feed may be triggered too often during the induction phase. In one embodiment, the preset value is from about 5% to about 20%, preferably 5% to 15%. In a specific embodiment, the preset value is 10% which means that the DO-stat setpoint is calculated as 10% plus the lowest DO level detected during the growth phase.

In a specific embodiment, the DO-stat setpoint is increased during the induction phase by about 2% when the pH decreases by ≧0.02 pH units or the DO-stat setpoint is decreased by about 2% when the pH increases by ≧0.02 pH units. This step can be repeated each time there is a change in the pH level.

The DO-stat setpoint is generally determined during the growth phase of the fermentation system. In one embodiment, the DO-stat setpoint is determined by a method comprising:

(1) adding cells and culture media to a fermentation system;

(2) measuring the dissolved oxygen (DO) and pH levels during the growth phase;

(3) adding nutrient feed to the system when there is an increase in the DO level and/or an increase in the pH level; and

(4) ceasing the nutrient feed when the DO levels are reduced, wherein a DO-stat setpoint is determined by adding a preset value to the lowest DO level measured during the growth phase.

The DO concentration of the fermentation medium is typically maximized at the beginning of the fermentation process. A person skilled in the art can readily determine the maximized DO concentration, which can vary based on a number of factors including, but not limited to, size and shape of the fermentor, type of cell, initial concentration of cells, type of product produced, type of fermentation medium and type of nutrient feed. In one embodiment, the DO concentration at the beginning of the induction phase is the same as the DO at the end of the growth phase.

The DO levels which are maximum at the start of the growth phase, decline shortly thereafter. Once the DO levels reach a predetermined value, the DO levels can be maintained at that value by increasing stirrer rate (in rpm) and airflow in a stepwise fashion until a maximum value. As such, the aeration to the system is not modified once it reaches the maximum value and it remains at that value for the remainder of the fermentation process. The predetermined value of the DO level that is used to trigger an increase in airflow and stirrer rate will be determined for each system and is based on the same factors used to determine the maximum DO concentration at the start of the system as described above (e.g. size and shape of fermentor, type of cell, initial concentration of cells, type of product produced, type of fermentation medium and type of nutrient feed). The maximum values for stirrer rate and airflow are predetermined based on the fermentation conditions and the size of the fermentor. For example, in a 120 L fermentor, maximum settings are about 200 rpm and 60 L/min for the rpm and airflow, respectively. For a 1200 L fermentor, maximum RPM and airflow can be 125 rpm and 564 L/min, respectively. These parameters can readily be determined by one of skill in the art. For example, stirrer rate (rpm) can be determined by the formula Tip speed (cm/min)=π×Di×RPM/60Di wherein Di is the diameter of the impeller. The airflow can be determined by the formula Volume of Air/Volume of Media (VVM)=airflow (L/min)/volume (L) of media.

Table 1 describes how the stirrer rate (rpm) and airflow can be increased during the growth phase. Once the maximum rpm and airflow values are reached, DO levels are allowed to fall indicating that the carbon sources in the medium are being depleted. Subsequent increases in DO and pH indicates that the carbon sources have been exhausted and nutrient feed is added. In a preferred embodiment, nutrient feed is added when there is an increase of ≧10% in the DO and/or an increase of ≧0.1 units in the pH. After the addition of the nutrient feed, the DO-stat setpoint can be determined. As mentioned previously, the DO-stat setpoint is determined by adding a preset value to the lowest DO level measured during the growth phase.

Throughout the growth phase, growth of the culture will be monitored, for example using OD₆₀₀, to determine when the growth phase is complete. For example, for the system described in the Examples, this is typically at an OD₆₀₀ of 20.

In a specific embodiment, the present invention provides a method for controlling the feeding of cells in a fermentation system comprising a growth phase and an induction phase, said method comprising:

(1) adding cells and culture media to a fermentation system;

(2) measuring the dissolved oxygen (DO) and pH levels during the growth phase;

(3) increasing the airflow and stirrer rate of the system to a maximum value when the DO reaches a predetermined value;

(4) adding a first nutrient feed to the system when there is an increase in the DO level and/or an increase in the pH level;

(5) ceasing the first nutrient feed when the DO levels are reduced wherein a DO-stat setpoint is determined by adding a preset value to the lowest DO level measured during the growth phase;

(6) measuring dissolved oxygen (DO) and pH levels in the fermentation system during the induction phase;

(7) (i) increasing the DO-stat setpoint when the pH falls below the pH-stat setpoint or (ii) decreasing the DO-stat setpoint when the pH increases above the pH-stat setpoint; and

(8) feeding the cells with a second nutrient feed when the DO level goes above the DO-stat setpoint.

To ensure the feed is not triggered or halted by rapid fluctuations around the DO-stat setpoint, the DO controller contains a dead band control for the DO setpoint that limits the cascade as a safety precaution.

The term “dead band control” is defined as the range through which the input may be varied without initiating a response. Usually expressed as a percentage of full-scale range. A dead band increment is built into the DO-stat controller. This is a specified increment (i.e. 0.5%) between the input % DO setpoint and the controller initiating or halting the feed. The interval set for the dead band in the automated system is user defined and can be selected as a system standard by observation of the range of % DO fluctuations of the manual system. In the manual system, the set dead band, is preprogrammed into the controller.

One of skill in the art will appreciate that all of the conditions of the fermentation system in both the growth and induction phase can be optimized for each application. The optimal conditions for a specific application will depend on a number of factors including, but not limited to, size and shape of the fermentor, type of cell, initial concentration of cells, type of product produced, type of fermentation medium and type of nutrient feed. Table 1 shows some of the standard parameters used in small (120 L) and large (1200 L) fermentors. The feed rate can be variable (e.g. for small fermentors) or fixed (e.g. for large fermentors). The culture media and nutrient feed can be purchased commercially or prepared by the user. The equipment including the fermentor and the probes for measuring DO and pH can also be obtained commercially and modified by the user if necessary.

Once the fermentation reaction is complete, the expressed protein can be harvested from the fermentation system using known techniques for example, as described in Example 1 and 2.

### (C) AUTOMATION

The steps of measuring the pH or DO concentration or adjusting the DO-stat setpoint or rate of nutrient feed can be done manually or be done by a computer or other machine. For example, the pH and/or the DO concentration can be monitored by a computer, or the DO-stat setpoint or rate of nutrient feed can be adjusted by a computer. Thus, the methods and the processes of the invention can be fully or partially automated.

Accordingly, the present invention provides an automated method for controlling the feeding of cells in a fermentation system comprising a fermentor, a pH probe, a dissolved oxygen (DO) probe, a feed pump and a programmable controller that controls the DO-stat setpoint and the feed pump wherein the method comprises:

(1) measuring the pH in the fermentor with the pH probe and i) if the pH increases above the pH-stat setpoint, decrease the DO-stat setpoint or ii) if the pH decreases below the pH-stat setpoint, increase the DO-stat setpoint; and

(2) measuring the DO level in the fermentor with the DO probe and adding nutrient feed from the feed pump when the DO levels go above the DO-stat setpoint.

The DO-stat setpoint and pH-stat setpoint can be determined for each system as described previously.

In a specific embodiment, the DO-stat setpoint is increased by about 2% when the pH decreases by ≧0.02 pH units or the DO-stat setpoint is decreased by about 2% when the pH increases by ≧0.02 pH units. This step can be repeated each time there is a change in the pH level.

In the automated method, the output readings of the pH probe and the DO probe are linked to a computer. The method of the present invention is especially suited for automation as it only requires two proportional feedback control loops. One control loop maintains the pH by using the output to adjust the DO-stat setpoint. The other control loop controls the DO by regulating the nutrient feed flow rate based on the adjusted DO-stat setpoint.

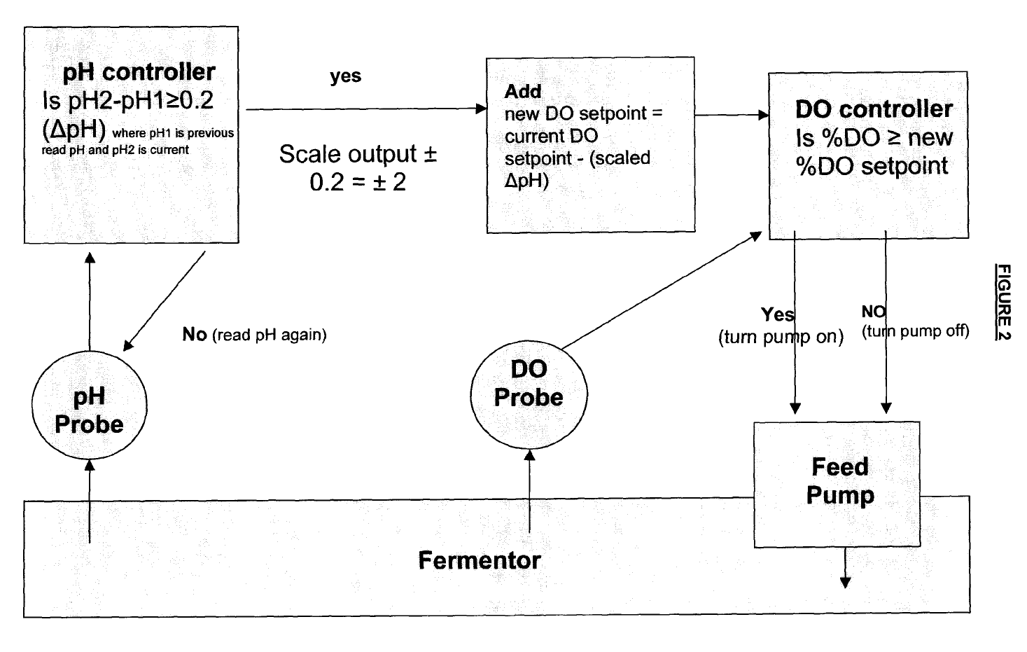

An algorithm was developed to constantly monitor pH and dissolved oxygen values in the fermentor and to implement the two proportional-integral feedback control loops as shown in FIG. 2. The first loop consists of the proportional or proportional-integral controller for pH control which determines whether to raise or lower the setpoint of the DO controller based on following the algorithm; if pH changed from the system designated value by 0.2 the % DO setpoint was proportionally but inversely changed by 2. The second loop consists of the % DO controller that determines whether the % DO within the system is at, above or below the setpoint. If the % DO increases past the set point the software returns a signal to the pump that initiates the addition of feed. The feed continues until the % DO in the system drops below the setpoint at which point a signal is sent to halt the addition of feed. The addition of feed in turn increases the metabolism of the cells and corresponding lowers the pH, which may then trigger a change in the % DO setpoint in the first loop. Correspondingly, ceasing of feed slows the cells metabolism which would result in an increase in the pH, which when detected by the pH sensor in the first loop would result in a signal to decrease the % DO setpoint, which would then initiate a feed through the second loop. Regulation of metabolism by the controlling the amount of feed and thereby the growth of the cells maintains tight pH control without the need for supplemental acid or base additions.

In one embodiment of the invention, the control loops continue until fermentation is complete. Fermentation can be completed by terminating fermentation at a defined point, including a defined time point, pH level, DO level, concentration of cells, concentration of the product of interest produced, or letting fermentation continue until the cells in culture die.

In a specific embodiment, the fermentor was connected through its commercially OPC hardware interface to a personal computer running Labview™ based control Cascade software. Signals from the pH and DO controller were interpreted by the software through the hardware interface. The software then determined if adjustments were required to the DO stat setpoint, based on the pH, made any necessary adjustments and then evaluated whether an initiation of feed was required. A signal would then be sent to the computer controlled feed pump that is also attached to the system.

The present invention also provides an automated system for controlling the feeding of cells in a fermentor, said system comprising:

(a) a fermentor comprising cells and culture media;

(b) a feed pump that delivers nutrient feed to the fermentor;

(c) a pH probe that measures the pH in the fermentor and wherein the pH measurement is linked to a programmable controller that controls the DO-stat setpoint; and

(d) a DO probe that measures the DO in the fermentor and wherein the DO measurement is linked to a programmable controller that controls the feed pump.

The following non-limiting examples are illustrative of the present invention:

### EXAMPLES

### Example 1

### Fermentation 120 L Volume

E. coli was used as the expression system for this protein of interest. The fermentation for the protein of interest included a growth phase and a fed-batch induction phase.

### Preparation of Primary Inoculum and Seeding Culture

1. Obtained one vial of E. coli cells.
2. Obtained a 2 L flask containing 500 mL of 2xYT seed medium.
3. Wiped down all components with 70% Reagent Alcohol before starting any work in the biological hood. In a biological safety aseptically added 2.5 mL of 0.5% Tetracycline solution to the seed medium using a 5 mL pipette and stripettor.
4. Wiped the outside of the WCB vial with 70% Reagent alcohol and transferred to the biological safety cabinet.
5. Allowed the cells to thaw at room temperature for 10 minutes -20 minutes.
6. Using a 2 mL pipette and stripettor inoculated the medium with 1.5 mL of cell suspension from the vial. Mixed the cells and medium by gently swirling the flask 5 times.
7. Transferred the inoculated flask to an incubator shaker and set to 200 rpm±20 rpm and 25° C.±1° C.
8. Let the inoculum grow for 10 hr±1 hr. After 9 hours of growth removed a sample for optical density value.
9. Shut off shaker and removed Primary inoculum flask. Wiped down with 70% Reagent Alcohol.
10. Placed Primary inoculum flask into biological hood.
11. Used a 5 mL serological pipette to aseptically withdraw a 5 mL sample from the Primary inoculum flask and placed into a 15 mL centrifuge tube. Placed the flask back on the shaker and restarted.
12. Pipetted 0.5 mL of the sample (step 7.11) using a micropipetter and diluted with 4.5 mL of sterile 2xYT medium in a 15 ml sterile centrifuge tube using a 5 mL pipette and stripettor. Measured the OD₆₀₀ on the spectrophotometer which had been blanked with sterile 2xYT medium.
13. Measured the pH.
14. Repeated steps 9 to 13 within the specified growth time until the culture met the following specification, OD600=2.0-2.5. The Secondary Inoculum flasks were ready to be seeded.
15. Obtained three 2 L Erlenmeyer flasks each containing 500 mL of 2xYT seed medium (CPR 0306). Wiped down flasks with 70% Reagent Alcohol before starting any work in the biological safety cabinet. In biological safety cabinet aseptically added 2.5 mL of 0.5% Tetracycline solution to each flask using a 5 mL pipette and stripettor.
16. Inoculated each flask with 5 mL of culture (1%).
17. Transferred the inoculated flasks to an incubator shaker and set to 200±20 rpm and 25° C.±1° C.
18. Let the inoculum in the flasks grow for 7 to 8 hours, and checked the OD after 8 hours of growth time. Performed a Gram stain on the culture at an OD≧1.6. Monitored growth until an OD of 2.0-2.5 was achieved.
19. Shut off the shaker and removed one Secondary Inoculum flask, wiped down with 70% Reagent Alcohol. Placed inoculum flask into biological safety cabinet.
20. Using a 5 mL serological pipette aseptically withdrew a 5 mL sample from one inoculum flask and placed into a 15 mL centrifuge tube. Placed the flask back on the shaker and restarted.
21. Pipetted 0.5 mL of the sample (step 7.11) using a micropipettor and diluted with 4.5 mL of sterile 2xYT medium in a 15 ml sterile centrifuge tube using a 5 mL pipette and stripettor. Measured the OD₆₀₀ on the spectrophotometer which had been blanked with sterile 2xYT medium.
22. Measured the pH.
23. Performed a gram stain on the culture. Submitted 2.5 mL of culture broth in a sterile 15 mL centrifuge tube to QC for testing. If the Gram stain was negative proceeded with vessel inoculation.

### Inoculation of 120 L Working Volume Chemap Fermentor

1. Ensured the 120 L fermentor had been cleaned and prepared with media. Aseptically removed approximately 30-50 mL of TB media from the fermentor.
2. Calibrated the DO probe to 100%.
3. Once the OD₆₀₀ of the seed flasks were in the range of 2.0-2.5 measured 1200 mL±25 mL into the 2 L sterile Inoculum assembly bottle in biological hood. Aseptically attached the inoculation line to the headplate of the vessel through one of the injection ports and transferred the inoculum into the vessel via a Chemap peristaltic pump.

### Monitoring Growth Establishing the DO Set Point

Culture media: TB Media: 12 g Tryptone, 24 g yeast extract (YE), 2.31 g Potassium Phosphate Monobasic, 12.5 g Potassium Phosphate Dibasic, 5 g Glycerol per 1 L of reverse osmosis (RO) water, pH 7.0±0.1. Sterilized for 30 minutes at 121° C.

Glycerol feed (Feed 1): 250 g Glycerol to a final volume of 500 mL of RO water. Sterilized for 30 minutes at 121° C.

Equipment: The fermentation process was carried out in a validated 150 L Chemap fermentor (120 L working volume) consisting of a digital pH display and automated controller for regulating temperature and stirrer speed (rpm). The associated stand alone automated dissolved oxygen (DO) controller maintained control of the feeds.

Standard Parameters: Once the sterilization was complete, the mixing was set to the initial value of 146 rpm, and the air flow to the initial value of 24 L/min. The temperature to 25° C.±1° C. was maintained throughout the process. (Table 1) The fermentor was prepared 12-16 hours prior to inoculation to ensure that the dissolved oxygen and pH probes had stabilized at the fermentation operating temperature.

In-process tests and acceptance criteria: The sterility of the fermentation media was tested as an in-process control in order to provide assurance that the starting production medium was sterile.

The airflow (L/min) and mixing speed (rpm) ramping stages were monitored to ensure consistent bacterial growth and the metabolic state of the culture.

To ensure the consistency of the metabolic state of the culture, and thus a consistent production run, the OD₆₀₀ of the fermentation culture was monitored with an OD₆₀₀ acceptance criteria of 15-18 within 17±2 hours while maintaining a pH of 7.2±0.1.
1. Withdrew a 30 mL sample from the Fermentor and measured OD and pH after 5 mins.
2. Monitored the Dissolved Oxygen (DO %) in the fermentor. The selected DO set point for the growth phase was 41% as established for this system. Increased the airflow and RPM of the stirrer to maintain a minimum DO % of 41%±2% in a stepwise manner from an initial setting of 24 L/min and 146 rpm respectively until maximum final settings of 200 rpm and 60 L/min were achieved as established conditions for this system.
3. 30 mL of broth were withdrawn from the vessel at least every three hours to monitor pH and OD values.
4. After maximum airflow and RPM were reached growth of the culture was closely monitored for changes in DO % and pH. Exhaustion of the supplied media was indicated by a marked increase in DO (>10%) and an increase in the pH (>7.1). When these DO and pH values were achieved, the DO % controller is activated to add “Feed 1” (aqueous solution of 50% glycerol) into the culture to maintain the growth of the culture.
5. After the first initial feed, noted the lowest point to which the DO % drops
6. The DO set-point was calculated for the rest of the growth phase and the start induction phase by adding 10% to minimum DO value obtained during the growth phase after the initiation of feed 1 where the pH of the culture between 7.10 and 7.2.
7. Addition of Feed 1 was determined by this initial DO % set point for the rest of the growth phase

### Inducing Protein Production: Maintaining a Stable pH

Culture media: TB Media: 12 g Tryptone, 24 g YE, 2.31 g Potassium Phosphate Monobasic, 12.5 g Potassium Phosphate Dibasic, 5 g Glycerol, 0.133 mL Polyglycol P2000 (antifoam) per 1 L of RO water, pH 7.0±0.1. Sterilized for 30 minutes at 121° C.

Induction feed (Feed 2): 2400 g Glycerol Sterilized for 30 minutes at 121° C. Dissolved 800 g L-arabinose in 1800 mL RO water and added to cooled glycerol solution. Two batches of identical composition were prepared.

Equipment: The fermentation process was carried out in a validated 150 L Chemap fermentor (120 L working volume) consisting of a digital pH display and automated controller for regulating temperature and stirrer speed (rpm). The associated stand alone automated dissolved oxygen (DO) controller maintained control of the feeds.

Standard Parameters: Mixing was maintained at 200 rpm, and the air flow at 60 L/min. The temperature to 25° C.±1° C. was maintained throughout the process. The pH was maintained at 7.2±0.1 (Table 1).

In-process tests and acceptance criteria: The sterility of the fermentation media was tested as an in-process control in order to provide assurance that the starting production medium was sterile.
1. 30 mL of broth were withdrawn from the vessel every hour to obtain OD and pH values. Once the OD₆₀₀ of the production culture reached a value of 20±1, the fermentor feed was switched to “Feed 2” (aqueous solution of 50% glycerol containing 0.17% L-arabinose) to induce expression of the protein over a period of 46 hours.
2. Monitored the pH of the culture. A pH range of 7.2±0.1 should be maintained throughout the post induction phase. When the pH was observed to decrease by 0.02 units increase the DO % set point by 2 units. The DO set-point was modified throughout the feeding stage in order to maintain the pH of the culture in the correct range.
3. Feed addition was triggered when the culture DO reached above the DO setpoint and continual pulse addition of feed 2 occurred until the culture DO went below the setpoint
4. Feed rate of the pump was adjusted as specified in Table 1 specifically for this size of fermentor. The pump rate is adjusted during the post induction feed to aid in the control of the culture pH.
5. Samples were taken (60±10 mL of broth) at 0 18, 36, 42, and 46 hours post-induction time and were submitted to quality control (QC) for testing for product.
6. The culture was allowed to grow for 46 hrs±15 mins.

### Harvesting

1. Connect the harvest hose from the harvest valve of the fermentor to the pump inlet that feeds a centrifuge. Connect the hose from the outlet of the pump to the inlet of the centrifuge.
2. Connect the hose from the outlet of the centrifuge to the bottom of a heat exchanger system and then from the top of the heat exchanger to the top of the collection tank of a microfiltration system.
3. Connect a solids collection tank to the solids discharge on the centrifuge.
4. At harvest change the temperature set point to 4° C.
5. Start the centrifuge and wait 4 minutes for maximum rotation.
6. Open the harvest valve on the fermentor and start the pump at 25 Hz, at a discharge time of 10 min and outlet pressure set point of 75 psi.
7. Adjust the pressure in the centrifuge to 75 psi by adjusting the regulator valve on the output line of the centrifuge.
8. After 5 minutes intervals obtain 20 ml of centrifuged culture supernatant from the centrifuge outlet line and read the OD₆₀₀.
9. Collect the culture supernatant in a clean tank.
10. When broth has been pumped through the centrifuge, manually discharge and shut down the centrifuge.
11. Disconnect the lines and the drain the excess supernatant into the collection tank.
12. Start the microfiltration pump at 20 Hz.
13. Maintain the system inlet speed at 35±5 psi by adjusting the pump speed.
14. Collect the microfiltered supernatant in a clean tank.
15. When the volume of the supernatant is down to 2 L stop the microfiltration system.
16. Weigh the microfiltered supernatant. This is now ready for purification of the protein.

### Example 2

### Fermentation 1200 L Volume

E. coli was used as the expression system for this protein of interest. The fermentation for the protein of interest included a growth phase and a fed-batch induction phase.

### Preparation of Primary Inoculum and Seeding Culture

1. Obtained one vial of E. coli cells.
2. Obtained a 2 L flask containing 500 mL of 2xYT seed medium.
3. Wiped down all components with 70% Reagent Alcohol before starting any work in the biological hood. In a biological safety aseptically added 2.5 mL of 0.5% Tetracycline solution to the seed medium using a 5 mL pipette and stripettor.
4. Wiped the outside of the WCB vial with 70% Reagent alcohol and transferred to the biological safety cabinet.
5. Allowed the cells to thaw at room temperature for 10 minutes -20 minutes.
6. Using a 2 mL pipette and stripettor inoculated the medium with 1.5 mL of cell suspension from the vial. Mixed the cells and medium by gently swirling the flask 5 times.
7. Transferred the inoculated flask to an incubator shaker and set to 200 rpm±20 rpm and 25° C.±1° C.
8. Let the inoculum grow for 10 hr±1 hr. After 9 hours of growth removed a sample for optical density value.
9. Shut off shaker and removed Primary inoculum flask. Wiped down with 70% Reagent Alcohol.
10. Placed Primary inoculum flask into biological hood.
11. Used a 5 mL serological pipette to aseptically withdraw a 5 mL sample from the Primary inoculum flask and placed into a 15 mL centrifuge tube. Placed the flask back on the shaker and restarted.
12. Pipetted 0.5 mL of the sample (step 7.11) using a micropipetter and diluted with 4.5 mL of sterile 2xYT medium in a 15 ml sterile centrifuge tube using a 5 mL pipette and stripettor. Measured the OD₆₀₀ on the spectrophotometer which had been blanked with sterile 2xYT medium.
13. Measured the pH.
14. Repeated steps 9 to 13 within the specified growth time until the culture met the following specification, OD600=2.0-2.5. The seeding fermentor was ready to be seeded.
15. Transferred 75 ml of 0.5% Tetracyline and 150 ml of seed culture to a 27 L Braun fermentor containing 15 L of 2xYT seed medium.
16. Allowed the culture to grow at 300±20 rpm, 12 L/min airflow and 25° C.±1° C.
17. Let the inoculum grow for 7 to 8 hours, and checked the OD after 8 hours of growth time. Monitored growth until an OD of 2.0-2.5 was achieved.
18. Aseptically withdrew a 30 mL sample.
19. Pipetted 0.5 mL of the sample (step 7.11) using a micropipettor and diluted with 4.5 mL of sterile 2xYT medium in a 15 ml sterile centrifuge tube using a 5 mL pipette and stripettor. Measured the OD₆₀₀ on the spectrophotometer which had been blanked with sterile 2xYT medium.
20. Measured the pH.
21. Performed a gram stain on the culture. Submitted 2.5 mL of culture broth in a sterile 15 mL centrifuge tube to QC for testing. If the Gram stain was negative proceeded with vessel inoculation.

### Inoculation of 1200 L Working Volume Fermentor

1. Ensured the 1200 L fermentor had been cleaned and prepared with media. Aseptically removed approximately 30-50 mL of TB media from the fermentor.
2. Calibrated the DO probe to 100%.
3. Once the OD₆₀₀ of the seed flasks were in the range of 2.0-2.5 the inoculum can be transferred to the production vessel. Aseptically attached the transfer line to the inoculation line on the 1200 L fermentor and to the harvest line on the 27 L fermentor.
4. The 27 L fermentor is pressurized to 8-10 psi and transferred 12 L from the 27 L fermentor to the 1200 L fermentor containing 1200 L of TB medium pH 7.0±0.1.

### Monitoring Growth Establishing the DO Set Point

Culture media: TB Media: 12 g Tryptone, 24 g yeast extract (YE), 2.31 g Potassium Phosphate Monobasic, 12.5 g Potassium Phosphate Dibasic, 5 g Glycerol per 1 L of reverse osmosis (RO) water, pH 7.0±0.1. Sterilized for 30 minutes at 121° C.

Glycerol feed (Feed 1): 250 g Glycerol to a final volume of 500 mL of RO water. Sterilized for 30 minutes at 121° C.

Equipment: The fermentation process was carried out in a validated 1500 L ABEC fermentor (1200 L working volume) consisting of a digital pH display and automated controller for regulating temperature and stirrer speed (rpm). The associated stand alone automated dissolved oxygen (DO) controller maintained control of the feeds.

Standard Parameters: Once the sterilization was complete, the mixing was set to the initial value of 53 rpm, and the air flow to the initial value of 240 L/min The temperature to 25° C.±1° C. was maintained throughout the process. (Table 1) The fermentor was prepared 12-16 hours prior to inoculation to ensure that the dissolved oxygen and pH probes had stabilized at the fermentation operating temperature.

In-process tests and acceptance criteria: The sterility of the fermentation media was tested as an in-process control in order to provide assurance that the starting production medium was sterile.

The airflow (L/min) and mixing speed (rpm) ramping stages were monitored to ensure consistent bacterial growth and the metabolic state of the culture.

To ensure the consistency of the metabolic state of the culture, and thus a consistent production run, the OD₆₀₀ of the fermentation culture was monitored with an OD₆₀₀ acceptance criteria of 15-18 within 17±2 hours while maintaining a pH of 7.2±0.1.
1. Withdrew a 30 mL sample from the Fermentor and measured OD and pH after 5 mins.
2. Monitored the Dissolved Oxygen (DO %) in the fermentor. The selected DO set point for the growth phase was 41% as established for this system. Increased the airflow and RPM of the stirrer to maintain a minimum DO % of 41%±2% in a stepwise manner from an initial setting of 240 L/min and 53 rpm respectively until maximum final settings of 123 rpm and 564 L/min were achieved as established conditions for this system.
3. 30 mL of broth were withdrawn from the vessel at least every three hours to monitor pH and OD values.
4. After maximum airflow and RPM were reached growth of the culture was closely monitored for changes in DO % and pH. Exhaustion of the supplied media was indicated by a marked increase in DO (>10%) and an increase in the pH (>7.1). When these DO and pH values were achieved, the DO % controller is activated to add “Feed 1” at 43 g/min (aqueous solution of 50% glycerol) into the culture to maintain the growth of the culture.
5. After the first initial feed, noted the lowest point to which the DO % drops
6. The DO set-point was calculated for the rest of the growth phase and the start induction phase by adding 10% to minimum DO value obtained during the growth phase after the initiation of feed 1 where the pH of the culture between 7.10 and 7.2.
7. Addition of Feed 1 was determined by this initial DO % set point for the rest of the growth phase.

### Inducing Protein Production: Maintaining a Stable pH

Culture media: TB Media: 12 g Tryptone, 24 g YE, 2.31 g Potassium Phosphate Monobasic, 12.5 g Potassium Phosphate Dibasic, 5 g Glycerol, 0.133 mL Polyglycol P2000 (antifoam) per 1 L of RO water, pH 7.0±0.1. Sterilized for 30 minutes at 121° C.

Induction feed (Feed 2): 2400 g Glycerol Sterilized for 30 minutes at 121° C. Dissolved 800 g L-arabinose in 1800 mL RO water and added to cooled glycerol solution. Two batches of identical composition were prepared.

Equipment: The fermentation process was carried out in a validated 1500 L ABEC fermentor (1200 L working volume) consisting of a digital pH display and automated controller for regulating temperature and stirrer speed (rpm). The associated stand alone automated dissolved oxygen (DO) controller maintained control of the feeds.

Standard Parameters: Mixing was maintained at 123 rpm, and the air flow at 564 L/min. The temperature to 25° C.±1° C. was maintained throughout the process. The pH was maintained at 7.2±0.1. Pump feed rate 43 g/min (Table 1).

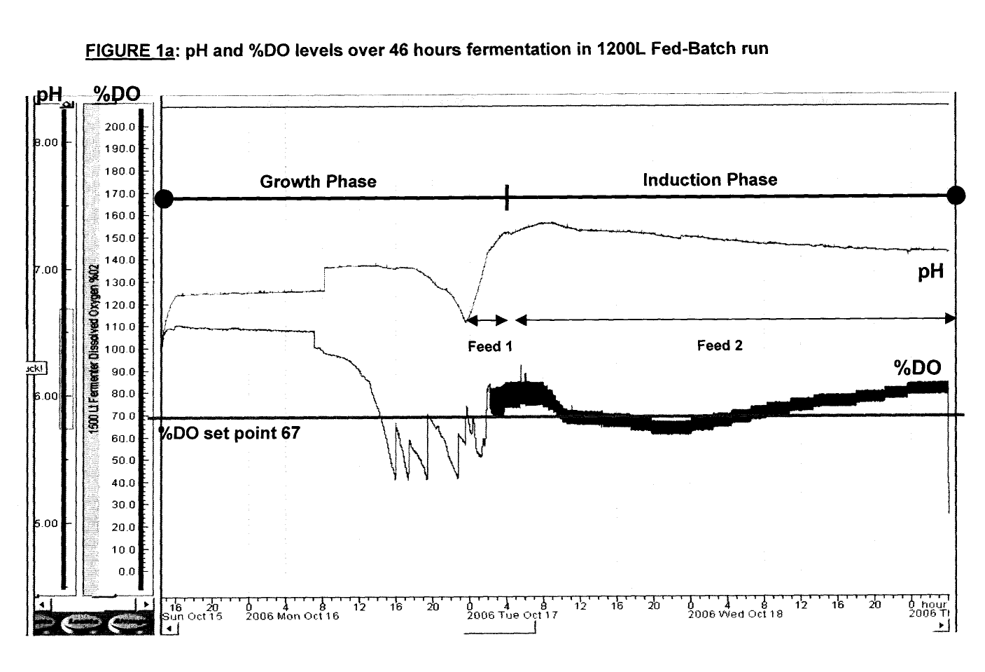

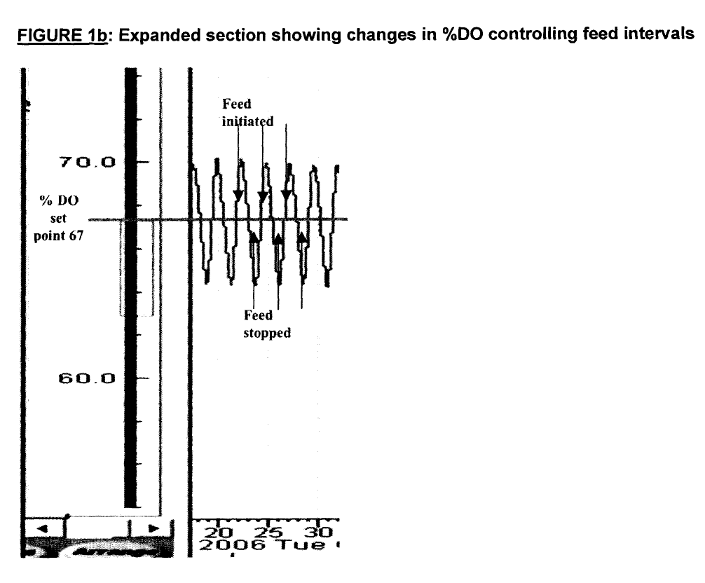

In-process tests and acceptance criteria: The sterility of the fermentation media was tested as an in-process control in order to provide assurance that the starting production medium was sterile.
1. 30 mL of broth were withdrawn from the vessel every hour to obtain OD and pH values. Once the OD₆₀₀ of the production culture reached a value of 20±1, the fermentor feed was switched to “Feed 2” (aqueous solution of 50% glycerol containing 0.17% L-arabinose) to induce expression of the protein over a period of 46 hours.
2. Monitored the pH of the culture. A pH range of 7.15±0.1 should be maintained throughout the post induction phase. When the pH was observed to increase/decrease by 0.02 units decrease/increase the DO % set point by 2 units. The DO set-point was modified throughout the feeding stage in order to maintain the pH of the culture in the correct range (FIG. 1).
3. Feed addition was triggered when the culture % DO reached above the % DO setpoint and continual pulse addition of feed 2 occurred until the culture % DO went below the setpoint (FIG. 1).
4. Feed rate of the pump maintained at 43 g/min specifically for this size of fermentor.
5. Samples were taken (60±10 mL of broth) at 0 18, 36, 42, and 46 hours post-induction time and were submitted to quality control (QC) for testing for product.
6. The culture was allowed to grow for 46 hrs±15 mins.

### Harvesting

1. Connect the harvest hose from the harvest valve of the fermentor to the pump inlet that feeds a centrifuge. Connect the hose from the outlet of the pump to the inlet of the centrifuge.
2. Connect the hose from the outlet of the centrifuge to the bottom of a heat exchanger system and then from the top of the heat exchanger to the top of the collection tank of a microfiltration system.
3. Connect a solids collection tank to the solids discharge on the centrifuge
4. At harvest change the temperature set point to 4° C.
5. Start the centrifuge and wait 4 minutes for maximum rotation.
6. Open the harvest valve on the fermentor and start the pump at 25 Hz, at a discharge time of 10 min and outlet pressure set point of 75 psi.
7. Adjust the pressure in the centrifuge to 75 psi by adjusting the regulator valve on the output line of the centrifuge.
8. After 5 minutes intervals obtain 20 ml of centrifuged culture supernatant from the centrifuge outlet line and read the OD₆₀₀.
9. Collect the culture supernatant in a clean tank.
10. When broth has been pumped through the centrifuge, manually discharge and shut down the centrifuge.
11. Disconnect the lines and the drain the excess supernatant into the collection tank.
12. Start the microfiltration pump at 20 Hz.
17. Maintain the system inlet speed at 35±5 psi by adjusting the pump speed.
18. Collect the microfiltered supernatant in a clean tank.
19. When the volume of the supernatant is down to 2 L stop the microfiltration system.
20. Weigh the microfiltered supernatant. This is now ready for purification of the protein.

### Example 3

### Automation

Manual intervention of the dissolved oxygen setpoint was used in the fermentation process of Examples 1 and 2. Specifically, the dissolved oxygen setpoint was manipulated manually when the pH changed due to cell growth following the algorithm if pH changed from the system designated value the % DO setpoint was proportionally but inversely changed where 0.2 change in pH=2% change in the % DO setpoint. The simplicity of this system allows for ease in automation.

A schematic of the system is shown in FIG. 2 which comprises a pH electrode, a dissolved oxygen electrode, a personal computer running OPC server software (hardware communications software), a personal computer running Labview™ Cascade feed controller software and a feed pump.

Cascade Controller software was developed to automate the % DO set point adjustment. The software has the advantage that the flow of data can be visualized and interpreted during execution (i.e. during fermentation). This results in a flexible and custom automation solution. An algorithm was developed to constantly monitor pH and dissolved oxygen values in the fermentor and to implement the two proportional-integral feedback control loops. The first loop consists of the proportional or proportional-integral controller for pH control which determines whether to raise or lower the setpoint of the DO controller based on following the algorithm; if pH changed from the system designated value by 0.2 the % DO setpoint was proportionally but inversely changed by 2. The second loop consists of the % DO controller that determines whether the % DO within the system is at, above or below the setpoint. If the % DO increases past the set point the software returns a signal to the pump that initiates the addition of feed. The feed continues until the % DO in the system drops below the setpoint at which point a signal is sent to halt the addition of feed. The addition of feed in turn increases the metabolism of the cells and corresponding lowers the pH, which may then trigger a change in the % DO setpoint in the first loop. Correspondingly, ceasing of feed slows the cells metabolism which would result in an increase in the pH, which when detected by the pH sensor in the first loop would result in a signal to decrease the % DO setpoint, which would then initiate a feed through the second loop. Regulation of metabolism by the controlling the amount of feed and thereby the growth of the cells maintains tight pH control without the need for supplemental acid or base additions.

LabView™ and National Instruments Industrial Automation OPC server software packages were used to create the controller software. Interaction of the controller and the fermentor can be done through the commercially available Lookout OPC server from National Instruments (for I/O with PLCs) or through National Instruments FieldPoint I/O bricks (4-20 mA I/O). This allows the same software to be scale-independent and simplifies technology transfer. See FIG. 2.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Standard System Established Parameters
Standard Parameters for 120 L and 1200 L Fed-Batch Fermentations

| Parameter | 120 L | 1200 L |
|---|---|---|
| Stirrer RPM | 1. 73 | 1. 53 |
| | 2. 97 | 2. 70 |
| | 3. 122 | 3. 80 |
| | 4. 146 | 4. 105 |
| | 5. 170 | 5. 123 (max) |
| | 6. 200 (max) | |
| Airflow | 1. 24 | 1. 240 |
| | 2. 32 | 2. 324 |
| | 3. 40 | 3. 396 |
| | 4. 48 | 4. 480 |
| | 5. 56 | 5. 564 (max) |
| | 6. 60 (max) | |
| pH | 7.15 ± 0.1 | 7.15 ± 0.1 |
| Temperature | 25° C. | 25° C. |
| Feed Rate (Post Induction) | 0 hrs 33 ± 1 | 43 ± 1 |
| | 3 hrs 25 ± 1 | |
| | 16 hrs 15 ± 1 | |
| | 23 hrs 11 ± 1 | |
| | 32 hrs 5 ± 1 | |

Note:
Stirrer RPM and Airflow are increased in stepwise intervals during the growth phase determined by a decrease in the % DO below 41%. These parameters can be calculated for different sized fermentors based on the formulas
VVM = airflow(L/min)/volume(L) VVM volume of air/volume of media
Stirrer rate Tip speed (cm/min) = π × Dᵢ × RPM/60 Di is the diameter of the impeller

TABLE 2
Fermentation Cultivation Conditions for Various Organisms

| Organism | Protein | pH | Temperature | References |
|---|---|---|---|---|
| Bacillus subtilis | Hyaluronic | 7.0 ± 0.4 | 37° C. | Widner et al. 2005 |
| | Acid | | | |
| Saccharomyces | Ergosterol | 5.5 ± 0.1 | 30° C. ± 1 | Shang et al 2005 |
| cerevisiae | | | | |
| Sulfolobus shibatae | | 3.5 | 75° C. | Krahae et al 1996 |
| Archaea | | 7.5 | 35° c. | Krahae et al 1996 |
| marinococcus | | | | |
| E coli | exotaxin | Not specified | 37° C. followed by | Suzuki et al 2006 |
| | | | 15° C. | |
| P. pastoris | Review of | 3-7 | 23-30° C. | Cos et al 2006 |
| | several | | | |

### REFERENCES

1. Chen W, Graham C, C, Carelli R B. Automated fed-batch fermentation with feed-back controls based on dissolved oxygen (DO) and pH for production of DNA vaccines. J. Ind. Microbiol. Biotechnol. 1997; 18:43-48.
2. Cos O, Ramon R, Montesinos J L, Valero F. Operational strategies, monitoring and control of heterologous protein production in the methylotrophic yeast Pichia pastoris under different promoters: A review. Microb. Cell Fact. 2006; 5:17.
3. Korz D J, Rinas U, Hellmuth K, Sanders E A, Deckwer W D. Simple fed-batch technique for high cell density cultivation of Escherichia coli. J. Biotechnol. 1995; 39:59-65.
4. Krahe M, Antranikian G, Markl H. Fermentation of extremophilic microorganisms. FEMS Microbiology Reviews 1996; 18:271-285.
5. Riesenberg D, Guthke R. High-cell-density cultivation of microorganisms. Appl. Microbiol. Biotechnol. 1999; 51:422-430.
6. Shang F, Wen S, Wang X, Tan T. High-cell-density fermentation for ergosterol production by Saccharomyces cerevisiae. J. Biosci. Bioeng. 2006; 101:3841.
7. Suzuki M, Roy R, Zheng H, Woychik N, Inouye M. Bacterial bioreactors for high yield production of recombinant protein. J. Biol. Chem. 2006.
8. Widner B, Behr R, Von D S et al. Hyaluronic acid production in Bacillus subtilis. Appl. Environ. Microbiol. 2005; 71:3747-3752.

1. A method for controlling the feeding of cells in a fermentation system during an induction phase, said method comprising
(a) measuring dissolved oxygen (DO) and pH levels in the fermentation system;
(b) (i) increasing a DO-stat setpoint when the pH falls below a pH-stat setpoint or (ii) decreasing a DO-stat setpoint when the pH increases above a pH-stat setpoint; and
(c) feeding the cells with nutrient feed when the DO level goes above the DO-stat setpoint.

2. The method according to claim 1, wherein the DO-stat setpoint is determined by a method comprising
(1) adding cells and culture media to a fermentation system;
(2) measuring the dissolved oxygen (DO) and pH levels during the growth phase;
(3) adding nutrient feed to the system when there is an increase in the DO level and/or an increase in the pH level; and
(4) ceasing the nutrient feed when the DO levels are reduced, wherein a DO-stat setpoint is determined as a preset value plus the lowest DO level measured during the growth phase.

3. The method according to claim 2 wherein the nutrient feed is added in step (3) when there is an increase of ≧10% in the DO level and/or an increase of ≧0.1 units in the pH level.

4. The method according to claim 2 wherein the stirrer rate and airflow levels in the fermentation system are increased prior to step 3) when the DO levels reach a predetermined value.

5. The method according to claim 4 wherein the stirrer rate and airflow are increased to a maximum value and maintained at that value for the remainder of the fermentation process.

6. The method according to claim 5 wherein the DO-stat setpoint is determined as 5% to 20% plus the lowest DO level measured during the growth phase.

7. The method according to claim 2 wherein the DO-stat setpoint is determined as 5% to 15% plus the lowest DO level measured during the growth phase.

8. The method according to claim 2 wherein the DO-stat setpoint is determined as 10% plus the lowest DO level measured during the growth phase.

9. The method according to claim 1 wherein the pH-stat setpoint is 6.7-7.5.

10. The method according to claim 9 wherein the pH-stat setpoint is 7.1-7.3.

11. The method according to claim 1 to wherein the cells are E. coli.

12. The method according to claim 1 wherein in step (b) the DO-stat setpoint is increased by about 2% when the pH decreases by ≧0.02 pH units or the DO-stat setpoint is decreased by about 2% when the pH increases by ≧0.02 pH units.

13. The method according to any claim 1, wherein the fermentation is fed-batch fermentation.

14. The method according to claim 1, wherein the nutrient feed comprises glucose, glycerol, other carbohydrates, and other nutrient solutions containing one or a combination of these sugars.

15. An automated method for controlling the feeding of cells in a fermentation system comprising a fermentor, a pH probe, a dissolved oxygen (DO) probe, a feed pump and a programmable controller that controls the DO-stat setpoint and the feed pump, wherein the method comprises:
(1) measuring the pH in the fermentor with the pH probe and i) if the pH increases above the pH-stat setpoint, decrease the DO-stat setpoint or ii) if the pH decreases below the pH-stat setpoint, increase the DO-stat setpoint; and
(2) measuring the DO level in the fermentor with the DO probe and adding nutrient feed from the feed pump when the DO levels go above the DO-stat setpoint.

16. An automated method according to claim 15 wherein in step (1) the DO-stat setpoint is increased by about 2% when the pH decreases by ≧0.02 pH units or the DO-stat setpoint is decreased by about 2% when the pH increases by ≧0.02 pH units.

17. An automated system for conducting the method according to claim 1, said system comprising:
(a) a fermentor comprising cells and culture media;
(b) a feed pump that delivers nutrient feed to the fermentor;
(c) a pH probe that measures the pH in the fermentor and wherein the pH measurement is linked to a programmable controller that controls the DO-stat setpoint; and
(d) a DO probe that measures the DO in the fermentor and wherein the DO measurement is linked to a programmable controller that controls the feed pump.

18. The automated system according to claim 17 wherein the pH probe and the DO probe are linked to a computer that controls the DO-stat setpoint and the feed pump.
